(19) United States
(12) Patent Application Publication
(10) Pub. No.: US 2003/0033018 A1
(43) Pub. Date: Feb. 13, 2003

(54) Patello-femoral joint arthroplasty

(76) Inventor: Alan C. Merchant, Mountain View, CA (US)

Correspondence Address: Paul J. Maginot, Maginot, Moore & Bowman, LLP, Bank One Center/Tower, 111 Monument Circle, Suite 3000, Indianapolis, IN, 46204-5115, US

(21) Appl. No.: 10/212,853

(22) Filed: Aug. 6, 2002

Related U.S. Application Data
(60) Provisional application No. 60/310,527, filed on Aug. 7, 2001.

(51) Int. Cl. A61F 2/38

(52) U.S. Cl. 623/20.19; 623/20.29

(57) ABSTRACT

A prosthetic patellar component includes a base and a bearing element. The base is operable to be affixed to an outer patellar surface. The bearing element includes first and second femoral engaging surfaces disposed between first and second edge surfaces and separated by a convex peak. The first and second edge surfaces include a gradual, or rounded, transition from a nearly posterior facing portion to a nearly superior or inferior facing portion, with adjacent surface portions having a relative angular displacement of less than about 30 degrees. The inferior-superior dimension is at least approximately 90% of the or medial-lateral dimension. The additional relative height and the gradually transitioning edges significantly reduce the likelihood of sudden posterior rotation during deep flexion movement. The patellar component may be employed in a PFJ system that engages a prosthetic femur implant that includes a trochlear groove. The femoral implant includes a medial bearing surface, a lateral bearing surface and a channel disposed therebetween. The femoral implant further includes a posterior surface having a maximum slope in medial-lateral cross-section of less than about 42 degrees to reduce the requirements for bone removal. The femoral implant also includes substantially mutually parallel outwardly projecting anchors that are configured for fixation within prepared bores in the femur.

### REFERENCE TO RELATED APPLICATION

This application claims priority to co-pending U.S. Provisional Application Ser. No. 60/310,527, entitled “Femoral Implant for Patello-Femoral Joint Arthroplasty and Associated Surgical Method”, filed on Aug. 7, 2001, in the name of the same inventors.

### FIELD OF THE INVENTION

The present invention relates generally to prosthetic patello-femoral joint assemblies, and more particularly, to individual components of such prosthetic assemblies and associated surgical methods of implantation.

### BACKGROUND OF THE INVENTION

The knee joint is a frequent place for joint damage, and the loss of normal (i.e. relatively pain-free) ambulatory function is a frequent result of such damage. Damage to the knee joint can occur as a result of any one of a plurality of causes, or a combination of causes. For example, a modest overextension of a knee weakened by osteoporosis can result in damage. Moreover, the extent of the damage to the knee joint can vary greatly depending on cause, age of the patient, pre-existing conditions and other factors.

The knee is a common source of problems because the joint has an unusually large range of motion and bears nearly half of the weight of the entire body. A primary knee movement, known as flexion-extension movement, includes the bending (flexion) and straightening (extension) of the leg in which a lower part of the leg (tibia and fibula bones) flex in relation to an upper part of the leg (femur bone) Ideally, the knee joint is capable of almost 180 degrees of flexion motion. The knee joint can also accommodate a certain amount of rotational motion in which the lower leg rotates a few degrees in relation to the upper leg.

This wide range of motion requires extensive contact surface between the femur and the tibia. The knee joint is rather loosely held together by tendons and ligaments to permit such a wide range of motion. The front or anterior side of the knee joint is protected by the knee cap or patella. The patella is held in place by ligaments and slides over a femoral joint surface during flexion movement. The patella and its ligaments are mechanically involved in joint extension. If any of the joint surfaces (femoral surface, patellar surface, or tibial surface) becomes damaged or roughened, the knee joint will not operate properly.

A common problem is damage to the patello-femoral joint that causes free motion of the patella to be inhibited and painful. Such damage is sometimes referred to as “runner's knee”. Patello-femoral joint (PFJ) damage can make normal joint movement almost impossible.

A variety of prosthetic replacements have been developed for different joint surfaces of the knee joint In extreme cases, the entire joint can be replaced with a prosthetic device Such a prosthetic replacement is referred to as a total knee replacement. However, total knee replacement requires a considerable time for recovery In less extreme cases it may be advantageous to replace only the damaged part of the joint.

In some cases, PFJ damage may be adequately addressed with a PFJ arthroplasty, as opposed to a total knee replacement system. This type of knee surgery is less drastic than total knee replacement. It is designed for patients whose main problems involve only the patello-femoral part of the knee and is directed to providing a smooth sliding relationship between the femur and the patella. The surface of the femur on which the patella slides is referred to as the trochlear groove. The trochlear groove is the indentation or groove located between the medial and lateral condylar surfaces at the inferior end of the femur.

In prior art PFJ prosthetic systems, a prosthetic patellar bearing surface is introduced. The prosthetic bearing surface typically includes an anchoring portion for receiving natural patellar remnants. As a result, the final patellar structure includes a posterior prosthetic bearing surface and an anterior natural patella surface. The anterior natural patella surface typically retains the connective tissue that connects the patella to the quadriceps and tibia.

In order to achieve adequate translational movement of the prosthetic patellar bearing surface, particularly in the presence of damage to the trochlear groove, a cooperating prosthetic femur implant is typically affixed onto the end, of the femur. The prosthetic femur implant in most cases includes a bearing surface that is specially adapted to receive the prosthetic patellar bearing surface to ensure reliable travel during flexion movement.

Such prior art systems, however, are typically highly artificial systems that employ unnatural patello-femoral tracking. One drawback of such systems is that they are not compatible with total knee replacement systems. In many cases, the PFJ system requires so significant an amount of bone removal as to render subsequent total knee replacement almost impossible.

More natural patellar devices employ a saddle-shaped design The saddle-shaped design may be used with or without a femoral implant and is intended to track the within the natural trochlear groove. While the current saddle-shaped designs track within the natural trochlear groove and/or implants that closely approximate the natural trochlear groove, it has been observed that designs of this nature can be prone to a phenomenon referred to as sudden posterior rotation.

Sudden posterior rotation sometimes occurs after a deep flexion movement in patients that have a weakened tendon condition known as patella infera In particular, as the knee is flexed farther and farther into acute flexion, it reaches a point where the patella suddenly rocks back over the sharp superior edges of the patella bearing. The patella bearing rotates around the transverse axis of the patella with the superior pole moving posteriorly and the inferior pole going anteriorly. Sudden posterior rotation often results in significant patient discomfort. Even without discomfort, the sudden posterior rotation can be annoying to the patient.

Another drawback of the prior art saddle-shaped patellar devices is that many require a femoral implant relatively deep trochlear groove to receive the peak edge of the saddle. Deep trochlear grooves also require relatively significant bone removal and thus render subsequent knee replacement difficult.

There is a need, therefore, for a patella prosthesis having the advantages of more naturally tracking designs but which is less prone to sudden posterior rotation. There is a further need for a femoral implant that requires less bone removal for implantation.

### SUMMARY OF THE INVENTION

The present invention address the above cited need, as well as others, by providing a prosthetic patellar bearing surface that includes first and second femoral engaging surfaces disposed between first and second edge surfaces, the first and second edge surfaces being rounded, or otherwise having a gradual transition from a nearly backward (or posterior) facing portion to a nearly vertical upward or downward (superior or inferior) facing portion. Moreover, the height (or inferior-superior) dimension is at least approximately 90% of the width (or medial-lateral) dimension. The additional relative height, as well as the rounded or otherwise gradually transitioning edges, significantly reduces the likelihood of sudden posterior rotation during deep flexion movement

A first embodiment of the invention is a prosthetic patellar component that includes a base and a bearing element. The base is operable to be affixed to an outer patellar surface. The bearing element comprises first and second femoral engaging surfaces that are separated by a convex peak. The first engaging surface extends medially from the peak and the second engaging surface extends laterally from the peak, the bearing element having a medial-lateral length and a largest inferior-superior length, wherein a ratio of the largest inferior-superior length is at least about 90% of the medial-lateral length. The first and second engaging surfaces are disposed between first and second edge surfaces, the first edge surface extending from a substantially posterior facing portion proximate to the first and second femoral engaging surfaces and a substantially vertical facing portion proximate the base. Adjacent medial-laterally extending surface portions of the first edge surface have an angular displacement less than about 30 degrees in the anterior-posterior direction.

Because the edge surfaces include adjacent surface portions having an angular displacement of less than about 30 degrees, no abrupt corners at the edge are present The lack of abrupt corners reduces the likelihood of sudden posterior rotation and its associated discomfort. In a preferred embodiment, the edge surfaces are rounded, such that the adjacent surface portions are continuous tangential portions of the rounded edge surface. However, alternative embodiments may include discrete polygonal edge portions that simulate a rounded edge surface by employing less than 30 degree displacement between adjacent portions. The present invention may be employed in a PFJ system that engages a natural trochlear groove or a prosthetic femur implant that includes a trochlear groove.

Another aspect of the present invention is a femoral implant device for use with a prosthetic patella arrangement. The femoral implant device preferably requires a reduced amount of bone removal In one embodiment the femoral implant device for use in patello-femoral joint arthroplasty includes a medial bearing surface, a lateral bearing surface and a channel disposed between the medial bearing surface and the lateral bearing surface The channel extends generally transverse the medial-lateral direction The lateral bearing surface, the medial bearing surface and the channel form an anterior surface of the implant device. The femoral implant further includes a posterior surface, the posterior surface having a maximum slope in medial-lateral cross-section of less than about 42 degrees.

The slope limitation helps ensure that the implantation process will require relatively less bone removal. The medial and lateral bearing surfaces are preferably convex and of differing sizes, both of which provide for better tracking of the patellar device

In a further feature, the posterior face of the femoral implant includes outwardly projecting anchors that are configured for fixation within prepared bores in the femur. The anchors are substantially mutually parallel and aligned along the impaction direction for driving the femoral implant into the femur.

The above-described features and advantages, as well as others, will become more readily apparent to those of ordinary skill in the art by reference to the following detailed description and accompanying drawings

### BRIEF DESCRIPTION OF THE DRAWINGS

FIG. 1 shows a side fragmentary view of a knee joint in which an exemplary prosthesis arrangement according to the invention has been implanted, the knee joint in approximately 45 degrees of flexion;

FIG. 2 shows a side fragmentary view of a knee joint in which an exemplary prosthesis arrangement according to the invention has been implanted, the knee joint in approximately 120 degrees of flexion;

FIG. 3 shows a top plan view of an exemplary patella bearing prosthesis according to the present invention;

FIG. 4 shows a bottom plan view of the bearing element of the patella bearing prosthesis of FIG. 3, the bearing element separated from the base;

FIG. 5a shows a top plan view of the base of the patella bearing element of FIG. 3, the base separated from the bearing element;

FIG. 5b shows a side plan view of the base of the patella bearing element of FIG. 3, FIG. 6 shows a cutaway view of the bearing element of FIG. 4 taken along line VI-VI of FIG. 4;

FIG. 7 shows a cutaway view of the bearing element of FIG. 4 taken along line VII-VII of FIG. 4;

FIG. 8 shows a front plan view of a femoral implant for use in connection with the patella bearing element of FIG. 3;

FIG. 9 shows a top plan view of the femoral implant of FIG. 8,

FIG. 10 shows a side plan view of the femoral implant of FIG. 8;

FIG. 11 shows a cutaway view of the femoral implant of FIG. 8 taken along line XI-XI of FIG. 9

FIG. 12 shows a perspective view of a femoral implant template disposed on a femur in accordance with a surgical method according to the present invention;

FIG. 13 shows a side plan view of patellar tissue resection of a surgical method according to the present invention;

FIG. 14 shows a patella bearing template for use in connection with a surgical method according to the present invention;

FIG. 15 shows a plan view of the use of the patella bearing template of FIG. 14 in preparing the patellar tissue for receiving the patella bearing prosthesis of FIG. 3; and

FIG. 16 shows a plan view of the patellar tissue being affixed to the patella bearing prosthesis of FIG. 3 in a surgical method according to the present invention.

### DETAILED DESCRIPTION

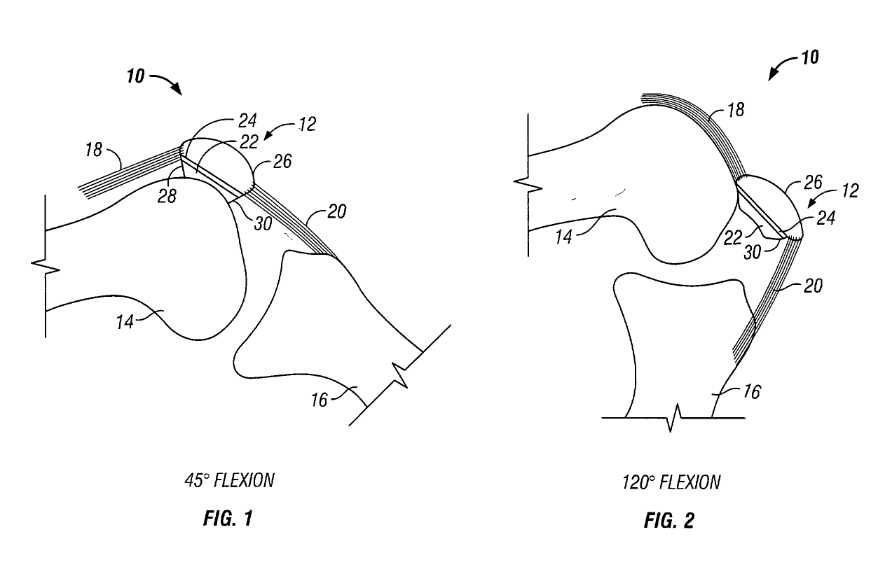

FIGS. 1 and 2 show side fragmentary views of a knee joint 10 in which an exemplary prosthesis arrangement 12 according to the invention has been implanted. FIG. 1 shows the knee joint 10 in approximately 45 degrees of flexion while FIG. 2 shows the knee joint 10 in approximately 120 degrees of flexion

In addition to the prosthesis arrangement 12, the knee joint 10 shown in FIGS. 1 and 2 includes a portion of a femur 14, a portion of a tibia 16, quadricep connective tissue 18 and a patellar ligament 20 The prosthesis arrangement 12 further includes a bearing element 22, a base 24 and natural patellar bone tissue 26. The bearing element 22 is secured to the base 24 such that partial rotation between the bearing element 22 and the base 24 may occur. The base 24 is securely affixed to the patellar bone tissue 26. The patellar bone tissue 26 is naturally affixed between the quadricep connective tissue 18 and the patellar ligament 20. In accordance with one aspect of the present invention, the bearing element 22 includes edge surfaces 28 and 30. At least the superior edge surface 28 has a gradual transition, for example, a rounded edge. As will be discussed further below, the superior-inferior dimension of the bearing element 22 is relatively large compare to prior art devices of like construct The prosthesis arrangement 12 moves or slides substantially in the inferior-superior direction during flexion motion of the knee. FIG. 2 illustrates a condition that may occur in patients having patella infera (weakened connective tissue). In particular, as the knee 10 moves to deep flexion as shown in FIG. 2 the weakened patellar ligament 20 allows the prosthetic arrangement to rotate slightly in the posterior direction. However, because of the relatively large inferior-superior dimension and the gradual transition of the superior edge surface 30, the prosthetic arrangement 12 may rotate smoothly back into position as the knee joint 10 moves out of deep flexion.

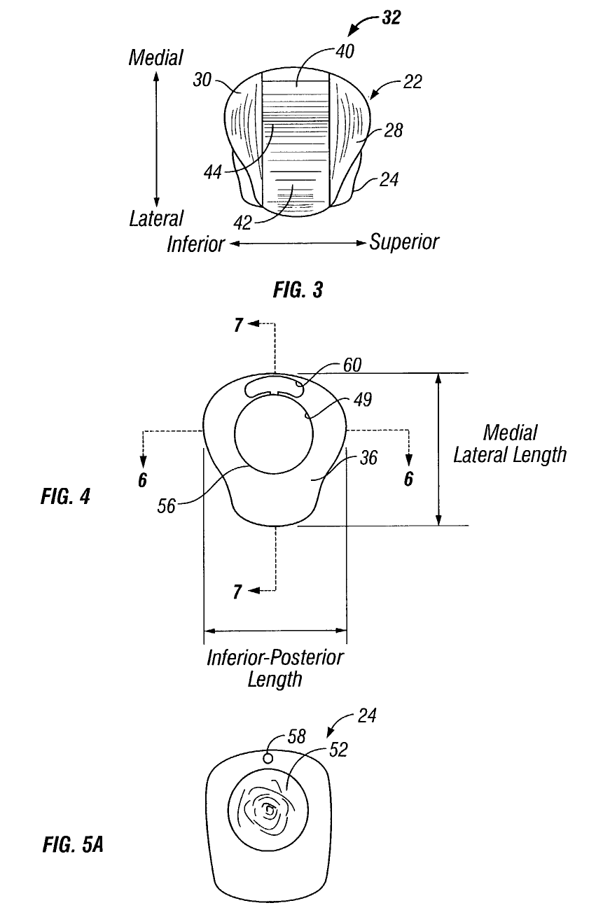

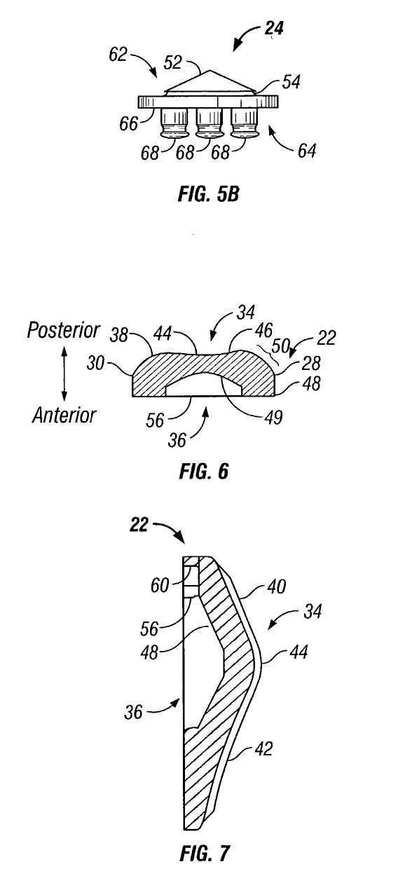

Further detail regarding an exemplary embodiment of the prosthesis arrangement 12 is provided in connection with FIGS. 3-7. FIG. 3 shows a bearing prosthesis 32 that includes the bearing element 22 and the base 24 FIGS. 4, 6 and 7 show different views of the bearing element 22 apart from the base 24, while FIGS. 5a and 5b show different views of the base 24 apart from the bearing element 22

With reference to FIGS. 3, 4, 6 and 7, the bearing element 22 includes a posterior side 34 and an anterior side 36. The posterior side 34 includes a bearing surface 38 defined by first and second femoral engaging surfaces 40 and 42. The first and second femoral engaging surfaces 40 and 42 are separated by a peak surface 44. The surfaces 40, 42 and 44 preferably cooperate to form an asymmetric saddle-type surface. To this end, the first femoral engaging surface 40 extends medially away from the peak surface 44, also sloping in the anterior direction as it extends medially away from the peak surface 44. Analogously, the second femoral engaging surface 42 extends laterally from the peak surface 44. The second femoral engaging surface 42 also slopes in the anterior direction as it extends laterally away from the peak surface 44.

In a preferred embodiment discussed herein, the sagittal cross-section (e.g. FIG. 6) of the peak surface 44 is concave, forming a slightly U-shaped channel. Likewise, the first and second femoral engaging surfaces 40 and 42 have similarly shaped sagittal cross-sections

The first and second engaging surfaces 40 and 42 are thus disposed end to end (i.e. serially) in the medial-lateral direction, with the peak surface 44 forming an intersection The first and second engaging surfaces 40and 42 further co-extend width-wise along the inferior-superior dimension. Also extending medial-laterally and bordering the inferior edges of the first engaging surface 40, the second engaging surface 42 and the peak surface 44 is the superior edge surface 28 Extending medial-laterally and bordering the superior edges of the first engaging surface 40, the second engaging surface 42 and the peak surface 44 is the edge surface 30

With particular reference to FIGS. 3 and 6, the superior edge surface 28 extends from a substantially posterior facing portion 46 (located proximate to the first and second femoral engaging surfaces 40 and 42) to a substantially vertical (superior) facing portion 48 proximate to the anterior side 36. Between the substantially posterior facing portion 46 and the substantially superior facing portion 48 is a gradually transitioning surface that may be considered to be divided into a plurality of adjacent medial-laterally extending surface portions. To ensure a gradual transition, it is preferable that the angle displacement between any two adjacent surface portions be less than about 30 degrees as measured in the anterior-posterior direction (i.e. measured in the view shown in FIG. 6).

In the exemplary embodiment described herein, the first edge surface 28 includes a curved portion 50, thereby guaranteeing throughout such portion that the angle displacement between adjacent surface portions is always less than about 30 degrees The curved portion 50 extends downward until it encounters the substantially posterior facing portion 46. In the exemplary embodiment described herein, the substantially posterior facing portion 46 extends substantially straight in the posterior direction from the anterior side 36 to a portion of the arc of the curved portion 50 that is approximately 20-25 degrees from the inferior-superior line that intersects its radius. Accordingly, the angle displacement between the tangent at the end of the curved portion 50 and the substantially posterior facing portion 46 is also 20-25 degrees, consistent with the overall 30 degree limitation discussed above.

In some embodiments, it may not be practical to limit the angle displacement between adjacent portions of the edge surface to about 30 degrees throughout the entire edge surface 28. In such cases, it has been found that by at least providing a curved portion such as the curved portion 50 can assist is reducing the likelihood of sudden posterior rotation, even if the angle displacement between the end of the curved portion and the substantially superior facing portion exceeds about 30 degrees. In particular, as long as the curved portion 50 extends sufficiently outward in the superior direction with an appropriate radius of curvature, the effect shown in FIG. 2 may typically be achieved. For example, if the curved portion 50 extends in the superior direction such that it covers at least about 20 percent of the largest inferior-superior dimension of the bearing surface 38, and if the curved portion 50 has a radius of curvature that is less than one-half of the largest inferior-superior dimension of the bearing surface 38, then enough of a gradual transition surface is provided by the edge surface 28.

If the radius of curvature is too large in such an embodiment, then the resulting edge surface would have too sharp of a cutoff and would not represent a gradual transition surface sufficient to effectively eliminate sudden posterior rotation. Likewise, if the curved portion 50 does not extend sufficiently far in the superior direction before terminating in the substantially superior facing portion 48, then the resulting edge surface would not exhibit enough of a transition area to effectively reduce sudden posterior rotation.

In an acceptable alternative, the angle of transition between the end of the curved surface 50 and the substantially superior facing portion may be about 45 degrees or less if the curved portion 50 extends in the superior direction such that it covers at least about 20 percent of the largest inferior-superior dimension of the bearing surface 38. While 45 degrees of angular displacement on the edge is somewhat abrupt, the length and curvature of the curved portion 50 will generally provide an adequate transition surface.

In other embodiments, the gradual transition surface may be accomplished by individual, non-curved (in the posterior-anterior direction) portions that form a polygonal pseudocurve that extends from the substantially posterior facing portion 46 to the substantially superior facing portion 48, as long as the angle between the adjacent portion is less than about 30 degrees. In still other embodiments, the pseudocurve may have an angle of up to about 45 degrees with respect to the substantially superior portion if the pseudocurve extends to at least until about 20 percent of the largest inferior-superior dimension.

All of the above limitations stress the idea of a gradual, convex transition surface to reduce the likelihood of sudden posterior rotation of the prosthetic arrangement 12. Prior art devices typically employed abrupt corners, such as an 80-90 degree transition with an insignificantly rounded corner. Such abrupt corners could result in sudden posterior rotation because the superior surface of the corner surface could “catch” on the femur when the knee joint comes out of deep flexion.

Another aspect of the present invention that assists in the inhibiting sudden posterior rotation problems is the relatively large inferior-superior length as compared to the medial-lateral length. In particular, the medial-lateral length is typically dictated in part by the medial-lateral length of the natural patella. The medial-lateral length is preferably as large as is practical to ensure optimal tracking, while not exceeding the approximate medial-lateral length of the natural patella. By using a superior-inferior size that is, at its longest point, at least approximately 90%, and preferably at least approximately 92% of the medial-lateral length, a transition edge surface (i.e. the edge surface 28) of significant length may be provided without sacrificing the inferior-superior dimensions of the femoral engaging surfaces 40 and 42.

The combination of the gradual transition surfaces and increased inferior-superior dimension thus provide good tracking, adequate contact surface, and inhibition of sudden posterior rotation during deep flexion of the knee. Such advantages of the prosthetic arrangement 12 are further enhanced because the arrangement is configured to allow for partial rotation of the natural patella tissue 26 with respect to the bearing element 22. To this end, the base 24 is configured to be attached to the bearing element in such a manner as to allow for partial relative rotation As a result, when the natural patella tissue 26 is affixed to the base 24, the natural patella tissue 26 may rotate in a limited way with respect to the bearing element 22, which more closely mimics the natural range of motion of a healthy knee joint.

Referring to FIGS. 4, 5a, 5b and 6, the anterior side 36 of the bearing element 22 includes a recess 48 which is configured to receive a corresponding bearing 52 of the base 24 The corresponding bearing 52 may rotate within the recess. The recess 48 in the exemplary embodiment described herein has the shape of an elevated and inverted cone. Accordingly, the bearing 52 has the shape of an elevated cone such that the bearing fits into the recess 48. The bearing 52 includes an annular lip 54 that cooperates with a corresponding annular lip 56 of the recess to retain the bearing 52 within the recess after being press fit.

The anterior side 36 of the bearing element 22 further includes a rotation limiting channel 60 that is configured to receive a small protrusion 58 that is disposed on the base 24. The rotation limiting channel 60 is preferable arc-shaped to allow the protrusion 58 to move in an arc, thereby allowing rotation of the bearing element 22 with respect to the base 24. However, the limits of the arc are chosen such that they correspond to the desired limitation of rotational freedom

In general, the base 24 has a size and shape roughly correlated to the size and shape of a human patella. The base 24 includes a posterior side 62 on which the bearing 52 and the protrusion are located and an opposing anterior side 64. The anterior side 64 includes a relatively flat patella receiving surface 66 and a plurality of anchors 68 As will be discussed below the anchors 68 are received into drilled bores in the natural patella bone tissue 26 to assist in securing the base 24 to the bone tissue 26.

The base 24 and the bearing element 22 are press fit together such that the bearing 52 is received into the recess 48 and the small protrusion 58 is received in to the rotation limiting channel 60. The annular lips 54 and 56 retain the base 24 and the bearing element together. The rotation limiting channel 60 limits the relative rotational movement of the base 24 and the bearing element 22 by only allowing limited travel of the small protrusion 58 within the channel 60.

When the assembled bearing prosthesis 32 is secured to the natural patella bone tissue 26, the resulting prosthetic arrangement 12 is capable of relatively natural movement within the body. In particular, the first and second femoral engaging surfaces 40 and 42 are advantageously configured to engage relatively normal femoral condyles to allow sliding movement of the arrangement 12 within the condyles In a preferred embodiment, the femur is further prepared with a femoral insert or implant that is configured to receive the bearing prosthesis 32

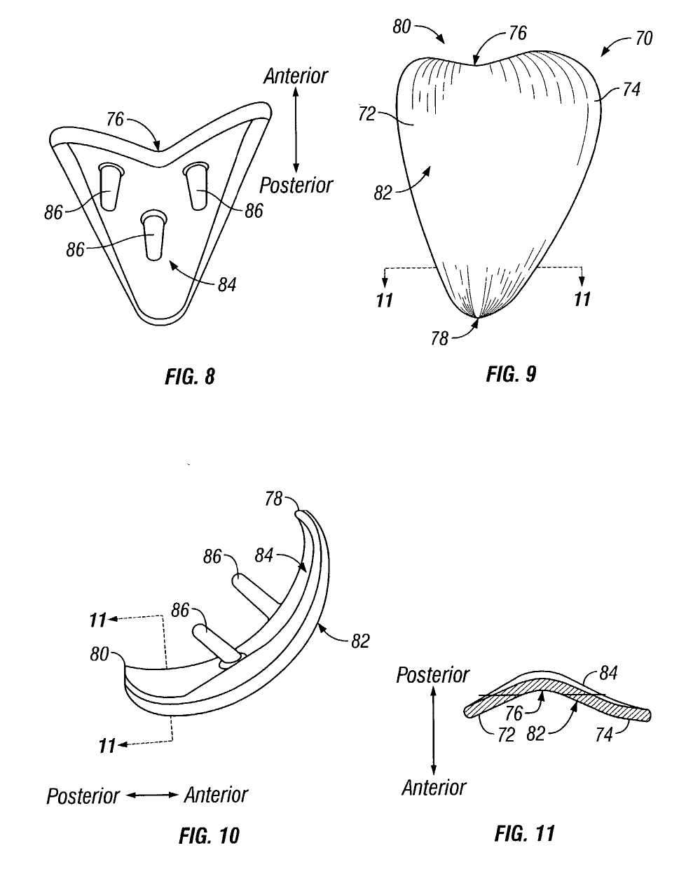

FIGS. 8, 9, 10 and 11 show an exemplary embodiment of a femoral implant 70 according to the present invention. Features of the femoral implant 70 include and asymmetrical wing shape that allows for better tracking of the asymmetrical bearing prosthesis 32 Another feature is the relatively shallow trochlear groove, which requires less bone removal prior to implantation. Requiring less bone removal provides the advantage of allowing subsequent procedures to be performed on the knee joint In particular, patients who have PFJ replacement are more likely to require a total knee replacement at some point in their lives. Accordingly, it is advantageous to limit the amount of bone removed during PFJ replacement in order to ensure that adequate femur bone tissue is intact for later implementation of the total knee prosthesis.

Referring now to FIGS. 8, 9 10 and 11, the femoral implant 70 includes a first (medial) condylar wing 72, a second (lateral) condylar wing 74, and a trochlear channel 76 that forms the intersection of the wings 72 and 74. The first condylar wing 72, the second condylar wing 74 and the trochlear channel 76 all include anterior bearing surfaces that, as a group, define the anterior bearing surface 82 of the femoral implant 70

The first condylar wing 72 is roughly triangular shaped and is configured to mimic the curvature of a condyle of a human femur. To this end, the anterior surface of the first condylar wing 72 forms a convex crescent arc shape in inferior-superior dimension, thereby curving somewhat in the posterior direction at both the inferior end 78 and the superior end 80, as shown in FIG. 10. The posterior surface of the first condylar wing 72 is substantially complementary, and thus concave. In addition, the anterior surface of the first condylar wing 72 has a convex arc shaped defined through its medial-lateral dimension, as shown in FIG. 11.

The second condylar wing 74 has a similar shape as the first condylar wing 72, although the second condylar wing 74 is generally wider in the medial-lateral dimension than the first condylar wing 72 The trochlear channel 76 runs generally from the inferior end 80 to the superior end 78 and forms the intersection of the convex condylar wings 72 and 74.

In general, the femoral implant 70 is installed at the inferior end of the femur 16 such that the trochlear channel 76 aligns with the natural trochlear groove of the femur. As will be discussed below, the femoral bone tissue must be prepared to receive the femoral implant 70. In particular, the femoral bone tissue is shaped such that it conforms substantially to the posterior surface 84 of the femoral implant 70.

In accordance with the exemplary embodiment described herein, the depth of the groove defined by the trochlear channel 76 is advantageously configured to balance the need for reducing the amount of femoral bone tissue that must be removed and need for sufficient tracking of the bearing element 22 of the patella prosthetic arrangement 12. To this end, the posterior surface 84 of the femoral implant 70 has a maximum slope of less than approximately 40 to 42 degrees, taken in any medial-lateral cross-section, such as is shown in FIG. 11. As a result, less femoral bone tissue need be removed from the vicinity of the trochlear groove than in prior art implants having a deeper (more severely sloped) channel. Preferably, the anterior bearing surface 82 has a complementary slope limitation.

The posterior surface 82 further includes a plurality of anchors 86 for securing the femoral implant to the femoral bone tissue. Each anchor 86 may suitably be a posteriorly extending member. As depicted in FIGS. 8 and 10, the anchors 86 are substantially parallel to each other. The anchors 86 are also generally perpendicular to a plane tangent to the femoral bone surface as prepared in accordance with the steps outlined below using the implant template 88.

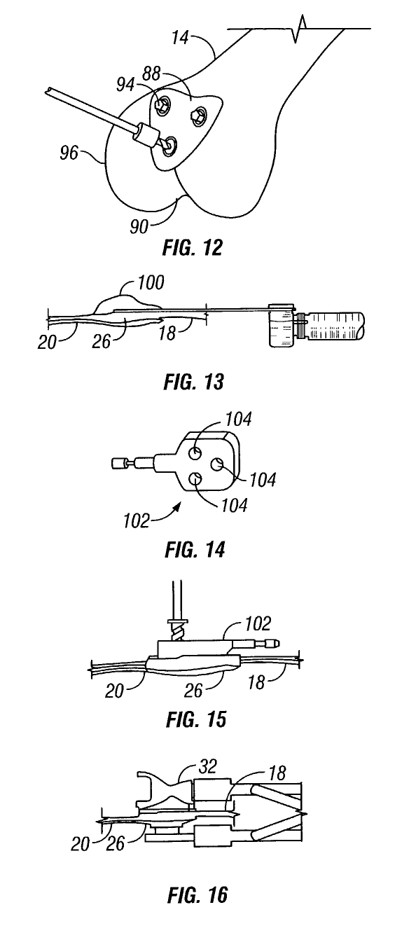

A process for performing a PFJ replacement employing the prosthetic patellar arrangement 12 and the femoral implant 70 is discussed with reference to FIGS. 12 through 16. Initially, it is advisable to review x-rays of the knee joint to determine which of a plurality of sizes should be employed. In general, the bearing element 22 is preferably available in four or five sizes ranging from 1.015 inches (inferior-posterior) by 1.126 inches (medial-lateral) to 1.520 inches (inferior-posterior) by 1.615 inches (medial-lateral). The femoral implant 70 is preferably available in four or five corresponding sizes ranging from 1.51 inches (inferior-posterior) by 1.18 inches (medial-lateral) to 2.4 inches (inferior-posterior) by 1.7 inches (medial-lateral).

Routine total joint arthroplasty protocols should be followed. The incision should be a midline skin incision, unless previous surgical scars indicate otherwise. A lateral retinacular release is performed up to but not including the superior lateral geniculate artery. If a more extensive release is necessary, it should be dissected and preserved for patellar blood supply. The patella should be dislocated and everted laterally.

Once the patella has been laterally dislocated, the trochlear groove and surrounding femoral surfaces must be prepared to receive the femoral implant 70. To this end, an implant template 88 is employed. FIG. 12 shows the implant template 88 fitted to the trochlear groove 90 of the femur 14. The implant template 88 has a shape that is substantially similar to that of the femoral implant 70, except that the implant template includes drill guides or drill bosses 94 instead of, and in the same position as, the anchors 86.

The implant template 88 is first aligned within the trochlear groove 90 as shown in FIG. 12 (however, alignment occurs without the drill bit 96 shown in FIG. 12). Once the template 88 is properly aligned, the outline of the template is marked on the cartilage and bone using a marking pen, knife or the like It is noted that the inferior end should not protrude into the intercondylar notch, but instead should be just proximal to the notch as shown in FIG. 12

The cartilage within the outline should be sharply resected High-speed burrs having small sharp osteotomes at the edges should be used to cut away a small portion of the subchondral bone within the outline. The implant template 88 is then placed into the groove again An outline is drawn again, and further cuts may be made if the implant template 88 is not yet flush with the articular cartilage surface The outline and cut steps may be repeated until the implant template 88 lays flush. Care should be taken to remove only small layers at a time to avoid the possibility of significant over-removal.

When the implant template 88 is flush, the components of the inferior end 78 of the femoral implant 70 will be flush, thereby reducing the possibility of overhang in which the prosthetic patellar arrangement 12 could get caught during deep flexion. By contrast, the portion of the wings 72 and 74 proximal the superior end 80 may protrude anteriorly from the bone without substantial ill effect.

After the trochlear cavity is created as discussed above and the implant template 88 fits properly, the implant template 88 may be used to drill holes in the femur 14 in which the anchors 86 will be received. This process is illustrated in part by FIG. 12. Once the holes have been drilled the femoral implant 70 is implanted. To this end, the anchors 86 are aligned with the drilled holes and an impacting device is used to drive the anchors 86 into the holes and the implant 70 into the cavity of the femur 14. Since the anchors are mutually parallel and generally perpendicular to the tangent plane to the prepared femur, the anchors can be readily driven along the impaction direction directly into the bone.

After the femoral implant 70 is in position, the patellar prosthetic arrangement 12 is prepared. To this end, the synovial tissue must be freed from the periphery of the patella down to the plane of the quadriceps and patellar tendon reflections. As shown in FIG. 13, the patellar articular surface 100 is resected parallel to and on the level of the quadriceps tendon connective tissue 18, thereby leaving the natural patella anterior bone tissue 26 connected to both the tissue 18 and the tibial ligament 20 The resection may suitably be performed using a patellar resection guide and an oscillating saw, not shown Suitable devices are commercially available.

Once the patella articular surface 100 is removed, a template 102 is used to drill the holes in the remaining bone tissue 26 for receiving the anchors 68 of the base 24 of the bearing prosthesis 32. (See FIGS. 1 and 5b). As shown in FIG. 14, the patellar template 102 includes three drill bosses 104 that are in the same configuration and alignment as the anchors 68 of the bearing prosthesis 32. The patellar template 102 otherwise has a shape and size similar to that the remaining bone tissue 26. FIG. 15 illustrates use of the patellar template 102 to drill the holes.

Thereafter, the bearing prosthesis 32 is pressed onto the remaining bone tissue 26 such that the anchors 86 are received into the drilled holes The resulting prosthetic arrangement 12 then includes the base 24, the bearing element 22 and the natural patellar bone tissue 26. However, the prosthetic arrangement 12 and the femoral implant 70 have only been prepared for trial reduction. To perform the trial reduction, the knee joint 10 is put through a full range of motion.

During the full range of motion, patellar excursion should be checked. If the patellar prosthetic arrangement 12 must be held in place with a thumb, then the alignment is not proper. Proper alignment of the extensor mechanism is important because the femoral implant 70 has a relatively deep anatomic sulcus. As a guideline, if the Q-angle is less than about 20 degrees, then a slightly larger lateral release will usually suffice. If the Q-angle is over 20 degrees, then a medial tibial tubercle transfer to a Q-angle of about 10 degrees should be considered. The Q-angle is measured intraoperatively with the knee extended and the limb rotated to that the patella is straight up and reduced into the trochlear channel 76.

The travel of the arrangement 12 should be checked to ensure that the bearing element 22 engages the trochlear channel 76 smoothly going from extension to flexion as well as going from flexion to extension. The travel of the arrangement 12 should also be checked to ensure that it does not catch at the inferior end 78 or superior end 80

If the trial reduction is successful, the prosthetic arrangement 12 may be finally assembled. To this end, the bearing prosthesis 32 is removed from the patellar bone tissue 26 and the femoral implant 70 is removed from the femur 14.

The trochlear area of the femur 14 is prepared using pulse lavage. After the femur dries, bone cement is applied to the posterior surface 84 of the femoral implant 70. The femoral implant 70 is then reinserted into the trochlear area of the femur 14 using an impact device, as discussed above. Excess cement should be removed The bearing prosthesis 32 is implanted onto the patellar bone tissue 26 using either a porous-coated implant or a cement technique. A patellar clamp 106 as shown in FIG. 16 may suitably be used to implant the bearing prosthesis 32. The resulting prosthetic arrangement should again be tested for proper excursion.

A number 0 braided polyester or a similar non-absorbable suture should be used for capsular closure, to allow for expedited range of motion for post-operative exercise.

It will be appreciated that the above described embodiments are merely exemplary, and that those of ordinary skill in the art may readily devise their own implementations of the present invention that incorporate the principles of the present invention and fall within the spirit and scope thereof.

It will further be appreciated that the shape of the bearing element is compatible with the LCS Total Knee system available from Depuy Orthopedics of Warsaw, Ind. Thus, if the patient subsequently (many years later) requires a total knee replacement, then the femoral implant 70 may be removed, and replace by the total knee system. The patellar prosthetic arrangement 12, however, need not be removed and may be used in conjunction with the total knee system

We claim:

1. A femoral implant device for use in patello-femoral joint arthroplasty, comprising.
a medial bearing surface;
an opposite lateral bearing surface;
a channel disposed between said medial bearing surface and said lateral bearing surface, the channel extending generally transverse to the medial-lateral direction of the device when the device is engaged to a femur, wherein said lateral bearing surface, said medial bearing surface and said channel form an anterior surface of the implant device; and
a posterior surface opposite said anterior surface configured for engaging the femur, the posterior surface having a maximum slope in medial-lateral cross-section of less than about 42 degrees.

2. The femoral implant device of claim 1 wherein said posterior surface is configured substantially similar to said anterior surface.

3. The femoral implant device of claim 1 wherein said medial bearing surface forms a convex curved surface in the medial-lateral direction.

4. The femoral implant device of claim 1 wherein said medial bearing surface forms a convex curved surface in the inferior-superior direction.

5. The femoral implant device of claim 1 wherein said medial bearing surface and bearing surface define different widths in the medial-lateral direction.

6. The femoral implant device of claim 5, wherein the width of said medial bearing surface is less than the width of said lateral bearing surface.

7. The femoral implant device of claim 1, wherein said posterior surface includes a plurality of substantially mutually parallel anchors projecting outwardly therefrom, each of said anchors configured for engagement within a prepared bore in the femur.

8. A femoral implant device for use in patello-femoral joint arthroplasty, comprising:
a medial bearing surface;
an opposite lateral bearing surface,
a channel disposed between said medial bearing surface and said lateral bearing surface, the channel extending generally transverse to the medial-lateral direction of the device when the device is engaged to a femur, wherein said, lateral bearing surface, said medial bearing surface and said channel form an anterior surface of the implant device; and
a posterior surface opposite said anterior surface configured for engaging the femur and including a plurality of substantially mutually parallel anchors projecting outwardly therefrom, each of said anchors configured for engagement within a prepared bore in the femur.

9. The femoral implant device of claim 8 wherein said posterior surface is configured substantially similar to said anterior surface.
